(19) United States
(12) Patent Application Publication
(10) Pub. No.: US 2024/0032479 A1
(43) Pub. Date: Feb. 1, 2024

(54) DEVICE FOR PROCESSING PLANT MATTER

(71) Applicant: Michael Taylor, Clinton Twp, MI (US)

(72) Inventor: Michael Taylor, Clinton Twp, MI (US)

(21) Appl. No.: 17/873,334

(22) Filed: Jul. 26, 2022

(51) Int. Cl. A01G 3/00; A01G 13/10

(52) U.S. Cl. CPC A01G 3/00; A01G 13/10

(57) ABSTRACT

A device and kit for processing plant matter. The device and kit comprise an insert having a generally tubular shape. The insert comprises agitation members extending from an inner surface of the insert, and for promoting detachment of the plant matter. The insert comprises openings allowing the detached plant matter to pass therethrough. The insert comprises anti-rotation members extending from an outer surface of the insert. Rotation and/or gyration of the insert relative to the longitudinal axis thereof, causes the plant matter to contact the agitation members.

### FIELD

The present teachings generally relate to a device for processing plant matter. The device can be operated manually.

### BACKGROUND

Some plant flowers contain secondary metabolites that are of commercial and pharmacological interest. Typically, these flowers are processed to remove extraneous plant matter such as leaves and stems in an effort to ultimately isolate or otherwise utilize the secondary metabolites. The extraneous plant matter can be trimmed away, such as by shearing. Trimming may be performed on flowers and extraneous plant matter that has been freshly harvested (e.g., about 1 day or less after harvesting), typically termed as wet trimming. One concern with this method is the buildup of tacky plant material on trimming equipment due to the moisture remaining in the plant material, which can require frequent cleaning of trimming equipment and thereby prolong the overall trimming process.

After harvesting, the flowers and extraneous plant matter can be dried (e.g., for about 5 days or more after harvesting) and then trimming performed thereafter, typically termed as dry trimming. Dry trimming can mitigate the buildup of tacky plant material as at least some of the moisture in the plant matter has been removed. Moreover, while wet trimming can be labor intensive and time consuming as it is typically performed with hand-held shears, drying of the plant matter can render plant matter (e.g., leaves) brittle relative to the plant matter at the time of harvest. Drying can allow for processing methods alternative to hand-shearing.

Dry trimming can be performed with automated mechanical equipment. Some automated equipment utilizes motor driven blades to cut away extraneous plant matter such as described in U.S. Application Publication Nos. 2021/0339264 A1 and 2013/0175372 A1. While this equipment is useful to process plant matter on a large scale, it can be cost-prohibitive for those who do so on smaller scales, such as those who grow and harvest plant matter at home.

Some plant species contain trichomes which bear the secondary metabolites of interest (e.g., in resinous form). There are devices and methods specifically directed to separation of trichomes, such as described in U.S. Pat. No. 10,507,223 B2. However, in some applications, it is desired to retain the trichomes and thus the secondary metabolites on the flower. Some techniques for the separation of plant matter such as leaves and stems can have the unintended consequence of removing at least some of the trichomes.

It would be desirable to provide a device and a kit therefor for processing plant matter.

It would be desirable to provide a device and a kit therefor for dry trimming plant matter.

It would be desirable to provide a device and a kit therefor that is cheaper to produce relative to automated mechanical equipment.

It would be desirable to provide and device and a kit therefor that can be manually operated.

It would be desirable to provide a device and a kit therefor that can separate plant matter without automated mechanical means.

### SUMMARY

The present disclosure relates to a device, which may address at least some of the needs identified above. The device may be employed for processing (e.g., dry trimming) plant matter. The device may comprise an insert. The insert may have a generally tubular shape.

The insert may comprise one or more agitation members (e.g., fingers and/or scalloped regions). The agitation members may extend from an inner surface of the insert. The agitation members may promote detachment of plant matter. The insert may be adapted, upon rotation and/or gyration relative to (e.g., around) the longitudinal axis thereof, to cause the plant matter to contact the agitation members.

The insert may comprise a plurality of openings. The openings may extend at least partially along the circumferential and/or longitudinal extent of the insert. The openings may allow the detached plant matter to pass therethrough.

The insert may comprise one or more anti-rotation members. The anti-rotation members may extend from an outer surface of the insert.

The present disclosure relates to a kit, which may address at least some of the needs identified above. The kit may be employed for processing (e.g., dry trimming) plant matter. The kit may comprise a container (e.g., 3.5-gal or 5-gal bucket). The kit may comprise an insert. The insert may have a generally tubular shape. The insert may be locatable into the container. The kit may optionally comprise a lid. The lid may seal the container.

The insert may comprise one or more agitation members (e.g., fingers and/or scalloped regions). The agitation members may extend from an inner surface of the insert. The agitation members may promote detachment of the plant matter. The insert may be adapted, upon rotation and/or gyration relative to (e.g., around) the longitudinal axis thereof, to cause the plant matter to contact the one or more agitation members.

The insert may comprise a plurality of openings. The openings may extend at least partially along the circumferential and/or longitudinal extent of the insert. The openings may allow the detached plant matter to pass therethrough.

The insert may comprise one or more anti-rotation members. The anti-rotation members may extend from an outer surface of the insert. The anti-rotation members may engage the container (e.g., frictionally) to inhibit rotation of the insert within the container. The anti-rotation members may maintain a gap between the insert and the container.

### BRIEF DESCRIPTION OF THE DRAWINGS

FIG. 1 is an exploded view of the device of the present teachings.

FIG. 2A is a top-down plan view of an insert of the present teachings.

FIG. 2B is a side plan view of an insert of the present teachings.

FIG. 2C is a sectional view of an insert of the present teachings.

FIG. 3 is an exploded view of the device of the present teachings.

FIG. 4A is a side plan view of an insert of the present teachings.

FIG. 4B is a top-down plan view of an insert of the present teachings.

### DETAILED DESCRIPTION

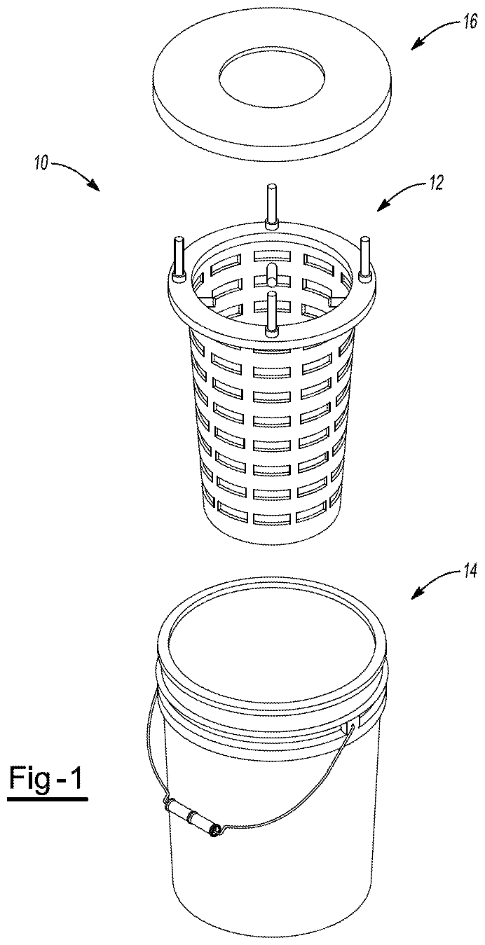

FIG. 1 illustrates a device 10 according to the present teachings. The device 10 comprises an insert 12 and a container 14 that receives the insert 12. A lid 16 seals the container 14 and retains the insert 12 therein. The device 10 is adapted to process (e.g., dry trim) plant matter. Plant matter can be located within the insert 12, the insert 12 is placed inside the container 14, and the lid 16 encloses the insert 12 and plant matter within the container 14. At least some extraneous plant matter that passes through openings 24 in the insert 12, discussed below, is captured within the container 14.

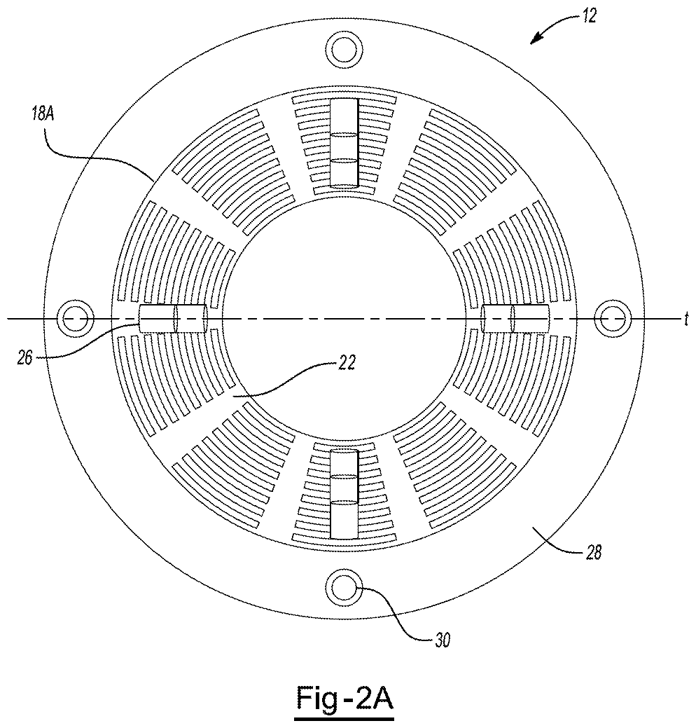

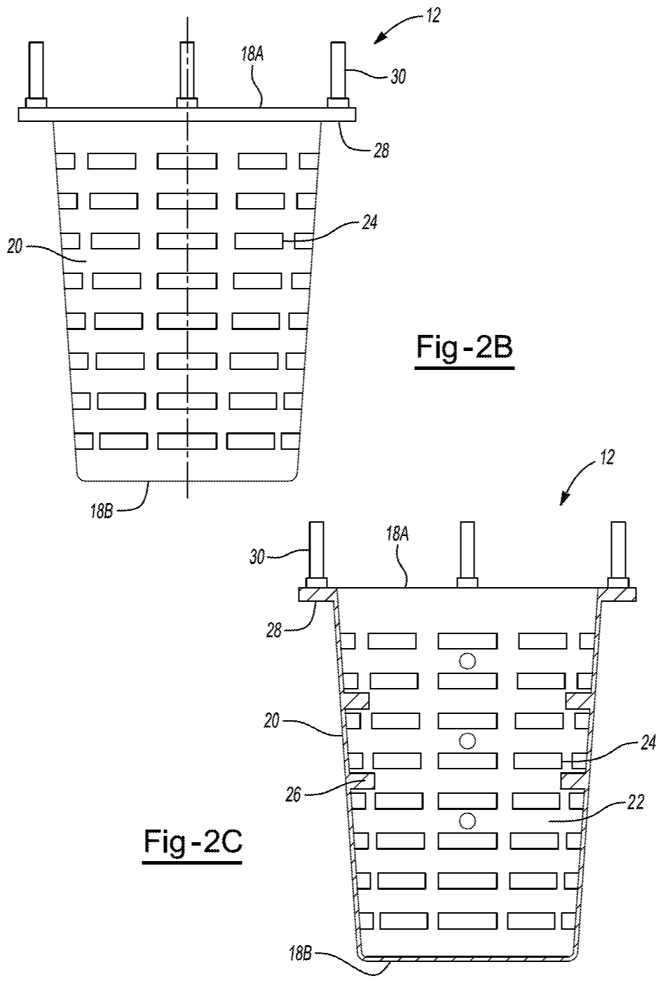

FIG. 2A through FIG. 2C illustrate an insert 12. The insert 12 comprises an open end 18A allowing for plant matter to be located within the insert 12. A closed end 18B opposes the open end 18A. The insert 12 is defined by a longitudinal axis l, extending through the open and closed ends 18A, 18B. The insert 12 is defined by a transverse axis t, oriented orthogonal to the longitudinal axis l.

The insert 12 has an outer surface 20 and an inner surface 22. Plant matter introduced to the insert 12 comes into contact with the inner surface 22. The insert 12 comprises a plurality of openings 24 located at least partially along the circumferential and longitudinal extent of the insert 12. The openings 24 extend from the outer surface 20 to the inner surface 22 such that extraneous plant matter can pass therethrough and exit the insert 12.

The insert 12 comprises a plurality of agitation members 26 in the form of fingers extending from the inner surface 22 of the insert 12. The agitation members 26 are located along the inner surface 22 in rows extending between the open and closed ends 18A, 18B of the insert 12. The agitation members 26 are located around the circumference of the inner surface 22. The inner surface 22, as referred to herein, may also be defined by a surface of the closed end 18B. The agitation members 26 are equally spaced along said circumference. The agitation members 26 promote the detachment of plant matter located within the insert 12. Rotation and/or gyration of the insert 12 relative to (e.g., around) the longitudinal axis/of the insert 12 causes the plant matter to contact the one or more agitation members 26 and extraneous plant matter is broken away from the subject plant matter.

The insert 12 comprises an anti-rotation member 28 in the form of a rim. The anti-rotation member 28 projects from an outer surface 20 of the insert 12. The anti-rotation member 28 extends around the entire circumference of the outer surface 20. The anti-rotation member 28 is located on the open end 18A of the insert 12. The anti-rotation member 28 circumferentially engages (e.g., frictionally engages) an inner surface of the container 14 to inhibit rotation of the insert 12 relative to the container 14. The anti-rotation member 28 also maintains a gap between the outer surface 20 of the insert 12 and the inner surface of the container 14.

The insert 12 comprises four anti-translation members 30 extending from the open end 18A of the insert 12. When the insert 12 is located within the container 14, the anti-translation members 30 span a gap between the open end 18A of the insert 12 and the lid 16 that is secured to the container 14. The anti-translation members 30 inhibit longitudinal axial translation of the insert 12 within the container 14.

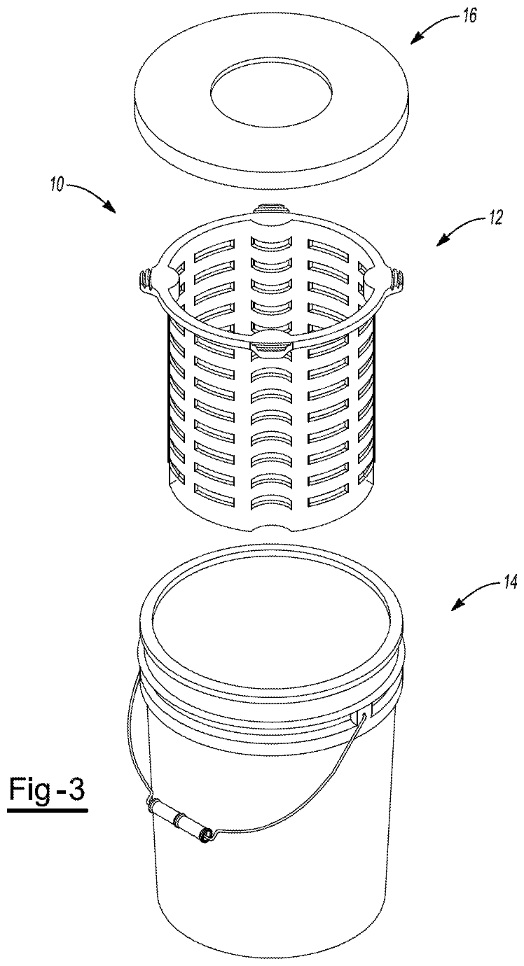

FIG. 3 illustrates a device 10 according to the present teachings. The device 10 comprises an insert 12 and a container 14 that receives the insert 12. A lid 16 seals the container 14 and retains the insert 12 therein. The device 10 is adapted to process (e.g., dry trim) plant matter. Plant matter can be located within the insert 12, the insert 12 is placed inside the container 14, and the lid 16 encloses the insert 12 and plant matter within the container 14. At least some extraneous plant matter that passes through openings 24 in the insert 12, discussed below, is captured within the container 14.

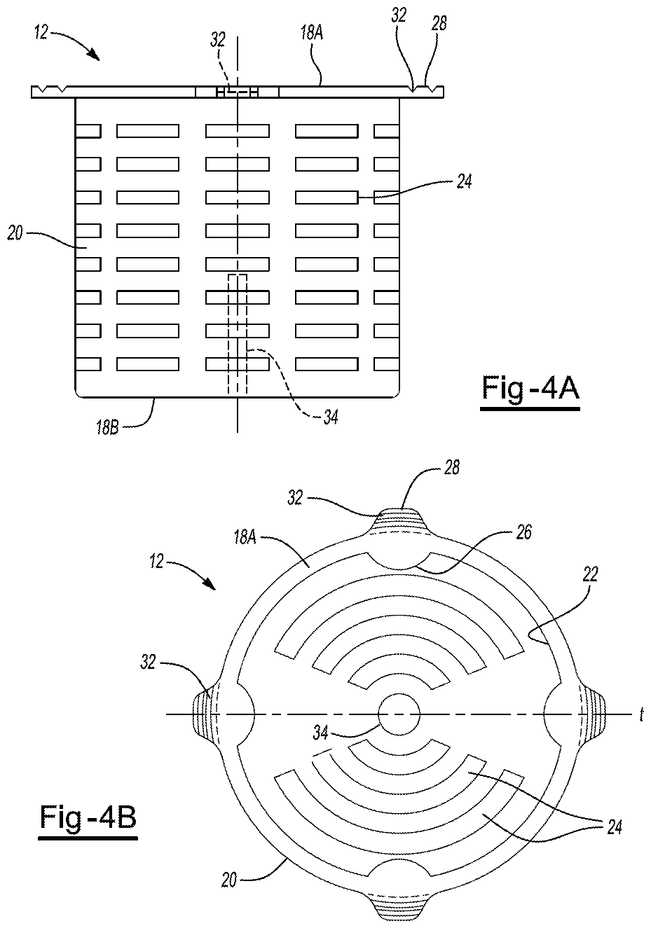

FIG. 4A and FIG. 4B illustrate an insert 12. The insert 12 comprises an open end 18A allowing for plant matter to be located within the insert 12. A closed end 18B opposes the open end 18A. The closed end 18B includes openings 24 for allowing plant matter to pass therethrough. The insert 12 is defined by a longitudinal axis l, extending through the open and closed ends 18A, 18B. The insert 12 is defined by a transverse axis t, oriented orthogonal to the longitudinal axis l.

The insert 12 has an outer surface 20 and an inner surface 22. Plant matter introduced to the insert 12 comes into contact with the inner surface 22. The insert 12 comprises a plurality of openings 24 located at least partially along the circumferential and longitudinal extent of the insert 12. The openings 24 extend from the outer surface 20 to the inner surface 22 such that extraneous plant matter can pass therethrough and exit the insert 12.

The insert 12 comprises four agitation members 26 in the form of scalloped regions defined by the inner surface 22 of the insert 12. The agitation members 26 extend between the open and closed ends 18A, 18B of the insert 12. The agitation members 26 are located around the circumference of the inner surface 22. The agitation members 26 are equally spaced along said circumference. The agitation members 26 promote the detachment of plant matter located within the insert 12. Rotation and/or gyration of the insert 12 relative to (e.g., around) the longitudinal axis l of the insert 12 causes the plant matter to contact the one or more agitation members 26 and extraneous plant matter is broken away from the subject plant matter.

The insert 12 comprises four anti-rotation members 28 in the form of discrete projections. The anti-rotation members 28 project from an outer surface 20 of the insert 12. The anti-rotation members 28 are equally spaced around the circumference of the outer surface 20. The anti-rotation members 28 are located on the open end 18A of the insert 12. The anti-rotation members 28 circumferentially engage (e.g., frictionally engage) an inner surface of the container 14 to inhibit rotation of the insert 12 relative to the container 14. The anti-rotation members 28 also maintain a gap between the outer surface 20 of the insert 12 and the inner surface of the container 14. The anti-rotation members 28 include two notches 32. The notches 32 provide bending points to facilitate the fitment of the insert 12 with the container 14 and/or accommodate containers 14 with different dimensions.

The insert 12 comprises a handle 34. The handle 34 is disposed in the center of the closed end 18B of the insert 12. The handle 34 provides a gripping surface for users to manipulate the insert 12 into and out of the container 14.

The insert 12, in lieu of anti-translation members 30 such as illustrated in FIG. 2B, has a length that is generally equal to the length between the bottom of the container 14 and the surface of the lid 16 that faces the bottom of the container 14, where the lid 16 is in a locked engagement with the container 14. The length of the insert 12 may be less than the length between the bottom of the container 14 and the surface of the lid 16, where the lid 16 is in a locked engagement with the container 14. Generally, the lid 16 may have a portion that extends a distance below the rim of the container 14. By virtue of the corresponding length, longitudinally axial translation of the insert 12 relative to the container 14 is mitigated.

The present disclosure provides for a device and kit therefor. The device and kit may be used for processing plant matter. The device and kit may be used for dry trimming plant matter. The plant matter may comprise subject plant matter and extraneous plant matter. The subject plant matter may include flowers. The subject plant matter may be retained within an insert. The extraneous plant matter may include stems and/or leaves. The stems and/or leaves may pass through the insert.

The device and/or kit may comprise an insert. The insert may function to receive plant matter, agitate plant matter, separate extraneous plant matter from subject plant matter, sift plant matter, or any combination thereof.

The insert may have a hollow body. The hollow body may have a generally tubular shape. Other hollow shapes (e.g., cuboid, rectangular prism, pentagonal prism, or the like) are contemplated by the present teachings. The shape may be oblong in one or more dimensions.

The insert may have two opposing ends. The insert may be defined by a longitudinal axis extending from a first end to a second end. A first end may be open, and a second end may be closed. The insert may have an inner surface defined by an inner dimension (e.g., diameter). The insert may have an outer surface defined by an outer dimension (e.g., diameter).

A dimension of the insert along a transverse axis thereof may be generally uniform from one end to the opposing end of the insert. That is, the walls of the insert may be generally parallel to the longitudinal axis of the insert.

A dimension of the insert along a transverse axis thereof may increase or decrease from one end to the opposing end of the insert. That is, the insert may taper from one end to the opposing end.

The insert may be fabricated from polymer, metal, or both. The polymer may include polyethylene, polypropylene, polyvinylchloride, polyamide, polycarbonate, polymethylmethacrylate, polyurethane, polyester, polyamide, polystyrene, the like, or any combination thereof. The polymer may include a filler (e.g., glass fibers and/or particulates).

The insert may be fabricated from casting, injection molding, material addition (e.g., 3D printing), stamping, drawing, or any combination thereof.

The insert may comprise one or more agitation members, one or more openings, one or more anti-rotation members, one or more anti-translation members, or any combination thereof.

The insert may comprise one or more agitation members. The agitation members may function to agitate plant matter, separate extraneous plant matter from subject plant matter, or both. Rotation and/or gyration of the insert may cause plant matter to roll and/or slide along the inner surface of the insert and contact the agitation members. Rotation and/or gyration of the insert may cause plant matter to contact other plant matter. Any contact with the inner surface of the insert, agitation members, and/or other plant matter may cause extraneous plant matter to break and/or tear away from subject plant matter.

Rotation and/or gyration may be relative to the longitudinal axis of the insert. The insert may be rotated around the longitudinal axis. The insert may be gyrated such that the longitudinal axis of the insert revolves around a parallel axis. The gyration may be along a generally circular and/or elliptical path. Grip of the container, lid, and/or insert by a user may be indexed around the circumference of thereof such that the user may grip the container, lid, and/or insert at one or more positions around the circumference thereof.

Typically, the plant matter may be dried prior to processing with the device of the present teachings. Drying my remove water from the plant matter. Drying may cause the plant matter to become more brittle relative to at the time of harvest. Thus, contact of the plant matter with the inner surface of the insert, the agitation members, and with other plant matter may cause extraneous plant matter to separate (e.g., break away) from subject plant matter.

Drying may take place over a period of about 1 day or more, 2 days or more, or even 4 days or more. Drying may take place over a period of about 14 days or less, 12 days or less, or even 10 days or less. The amount of time over which plant matter may be dried may be a function of temperature and/or humidity.

Drying may take place in an environment with a temperature of about 60° F. or more or even 65° F. or more. Drying may take place in an environment with a temperature of about 80° F. or less or even 75° F. or less.

Drying may take place in an environment with a humidity of about 40% or more or even 45% or more. Drying may take place in an environment with a humidity of about 60% or less or even 55% or less.

The agitation members may extend from the inner surface of the insert. The agitation members may extend cantilever into the hollow portion of the insert. The agitation members may extend toward the central longitudinal axis of the insert. The agitation members may extend at an angle relative to a transverse axis of the insert, the transverse axis being orthogonal to the longitudinal axis.

The agitation members may be defined by a dimension of about 0.5 inch or more, 1 inch or more, or even 1.5 inches or more. The agitation members may be defined by a dimension of about 3 inches or less, 2.5 inches or less, or even 2 inches or less. The dimension may extend from the inner surface of the insert to a terminal end of the agitation members.

The agitation members may be integrally formed to the insert. By way of example, the agitation members and insert may be molded together (e.g., injection molded). By way of another example, the insert and agitation members may be fabricated by material addition (e.g., 3D printing).

The agitation members may be fastened to the insert. The agitation members may be mechanically fastened, chemically fastened, or both. Mechanical fasteners may include screws, nails, rivets, the like, or any combination thereof. Chemical fasteners may include epoxy adhesive, acrylic adhesive, polyurethane adhesive, rubber adhesive, silicone adhesive, the like, or any combination thereof.

The agitation members may be one or more scalloped regions. The scalloped regions may be defined by radial segments formed in the inner surface of the insert. The radial segments may be defined by a radius of curvature. The radius of curvature may be about 2 cm or more, 4 cm or more, 6 cm or more, or even 8 cm or more. The radius of curvature may be about 16 cm or less, 14 cm or less, 12 cm or less, or even 10 cm or less. The radial segments may be defined by a chord length. The chord length may be about 2 cm or more, 4 cm or more, 6 cm or more, or even 8 cm or more. The chord length may be about 16 cm or less, 14 cm or less, 12 cm or less, or even 10 cm or less. The dimensions of the scalloped regions may be selected to provide adequate agitation but avoid excessive agitation.

The outer surface of the insert in the regions of the scalloped regions may form a corresponding radial segment devoid of material. In this regard, the wall thickness of the insert may be generally uniform around the entire insert.

The outer surface of the insert in the regions of the scalloped regions may be uniform with the rest of the outer surface. In this regard, the wall thickness of the insert in the regions of the scalloped regions may be greater than the wall thickness where the scalloped regions are not present.

The scalloped regions may be located around the inner surface of the insert. The scalloped regions may extend from a first end of the insert to a second end of the insert or at least partially therebetween. The scalloped regions may extend parallel to a longitudinal axis of the insert. The scalloped regions may be equally or unequally spaced relative to each other around the inner surface of the insert. Some of the scalloped regions may be equally spaced relative to each other while some other scalloped regions may be unequally spaced relative to each other.

The scalloped regions may be suitable for injection molding That is, the scalloped regions may be generally uniform along the length of the insert such that at least two portions of a mold can be released from each other and the insert removed from the mold.

The agitation members may be one or more fingers. The fingers may be defined by a first end coupled to or integrally formed with the inner surface of the insert and a second end opposing the first end, the second end being free ends.

The fingers may have a cylindrical shape. The fingers may have one or more corners and sides (e.g., a cuboid shape, rectangular prism shape, a pentagonal prism shape, or the like).

The fingers may be rigid. The fingers may be elastically deformable.

The fingers may be disposed around the inner surface of the insert. The fingers may be arranged in one or more rows along the circumferential extent and/or the longitudinal extent of the insert. Different rows of fingers may be offset by a dimension in the circumferential extent and/or the longitudinal extent from one another.

The scalloped regions may provide for a gentler agitation relative to fingers. The scalloped regions may remove fewer trichomes relative to fingers.

The insert may comprise one or more handles. The handle may function to provide a gripping surface for a user to manipulate the insert. The insert may be manipulated relative to the container. The handle may be a projection that extends from the inner surface and/or closed end of the insert. The handle may be located anywhere on the inner surface and/or closed end of the insert. The handle may be located on the center of the closed end of the insert.

The insert may comprise one or more openings. The openings may function to allow extraneous plant matter to pass therethrough, promote the separation of extraneous plant matter from subject plant matter, or both. The openings may sift plant matter. Plant matter having a dimension generally equal to or less than a dimension of an opening may pass therethrough. The openings may be sized such that flowers are retained within the insert.

The openings may cause the separation of plant matter. That is, extraneous plant matter still attached to the subject plant matter may pass through an opening and pry away from the subject plant matter as the insert is rotated and/or gyrated.

The openings may have a shaped profile. The profile may be square or rectangular. Other profile shapes are contemplated by the present teachings.

The openings may be located along the longitudinal and/or circumferential extent of the insert. The openings may be located on the closed end of the insert. While reference herein is made to a “closed end”, the present disclosure contemplates that the closed end may comprise openings while also comprising material in which the openings are defined, where at least some plant matter may be retained within the insert by the closed end.

The one or more openings may be arranged in one or more rows in the longitudinal and/or circumferential directions. One or more rows may be aligned with or staggered to one or more other rows.

The insert may comprise one or more anti-rotation members. The anti-rotation members may function to inhibit rotation of the insert within a container.

The anti-rotation members may extend from the outer surface of the insert. The anti-rotation members may contact a container and/or a lid. Due to frictional engagement of the anti-rotation members with the container and/or lid, rotation of the insert relative thereto may be mitigated.

The anti-rotation members may maintain a gap between the insert and the container. An open volume may be formed between the insert and the container. Extraneous plant matter may pass through the insert and collect within the open volume.

The container into which the insert is locatable may have a tapered inner diameter between two opposing ends of the container. The anti-rotation members may extend from the insert a distance to correspond with the inner diameter of the container defined at any point along the taper. The anti-rotation members may fit to the inner diameter of the container such that a gap is maintained between the closed end of the insert and the bottom of the container. In this regard, the bottom of the insert may be free of contact with the container. Only the anti-rotation members may engage the container.

The anti-rotation member may be a rim. The rim may extend around the entire circumference of the insert.

The anti-rotation members may be two or more projections. The two or more projections may each extend around a radial segment of the insert. The quantity, dimensions, locations, or any combination thereof of the projections may be provided such that the insert does not shift within the container. By way of example, two projections disposed in opposition to each other on the insert may allow for shifting of the insert in a direction transverse to the axis extending through the opposing projections. In this regard, three equally-spaced projections disposed around the outer surface of the insert may prevent shifting of the insert in all transverse directions.

The anti-rotation members may be bent, trimmed, broken, or the like. In this regard, the dimensions of the anti-rotation members may be manipulated to fit a container. Containers employed with the insert may have an inner diameter that is smaller than the combined diameter of the insert and anti-rotation members. Moreover, containers may be fabricated out-of-specification or within a pre-determined tolerance range. Thus, the anti-rotation members may be manipulated to fit within the container.

The anti-rotation members may be elastically deformable such that they can bend to fit in a container. The anti-rotation members may be plastically deformable such that a portion thereof can be broken away (e.g., by bending).

The anti-rotation members may be provided with one or more notches. The notches may form a weak point in the material to promote bending and/or breaking. The notches may be fabricated such that the anti-rotation members are thinner in the area of the notches relative to where the notches are absent. In this regard, the anti-rotation members may be cut along the notches. Plastically deformable anti-rotation members may comprise notches to promote a portion thereof with breaking away from the insert.

The insert may comprise one or more anti-translation members. The anti-translation members may function to inhibit translation of the insert along a longitudinal axis of a container.

The anti-translation members may be one or more projections. The anti-translation members may extend from one or both ends of the insert. The anti-translation members may span a gap between the open end and/or the closed end of the insert. The anti-translation members may contact a bottom of the container and/or the lid.

The anti-translation members may be integrally formed to the insert. By way of example, the anti-translation members and insert may be molded together (e.g., injection molded). By way of another example, the insert and anti-translation members may be fabricated by material addition (e.g., 3D printing).

The anti-translation members may be fastened to the insert. The anti-translation members may be mechanically fastened, chemically fastened, or both. Mechanical fasteners may include screws, nails, rivets, the like, or any combination thereof. Chemical fasteners may include epoxy adhesive, acrylic adhesive, polyurethane adhesive, rubber adhesive, silicone adhesive, the like, or any combination thereof.

The anti-translation members may be cut to a length to fit within the container when the lid is in locked engagement with the container. The anti-translation members may comprise one or more notches. The notches may form a weak point in the material to promote bending and/or breaking. The notches may be fabricated such that the anti-translation members are thinner in the area of the notches relative to where the notches are absent. In this regard, the anti-translation members may be cut along the notches. Plastically deformable anti-translation members may comprise notches to promote a portion thereof with breaking away.

The present disclosure contemplates that the length of the insert may generally correspond to the length between the bottom of the container and a surface of the lid facing the bottom of the container. In this regard, the relationship between the insert and the container and/or lid may be devoid of a space through which insert can axially translate. Thus, the insert may be free of anti-translation members. Generally, a portion of the lid may extend below the top rim of the container. In this regard, the lid may define a space into which the rim of the container fits into.

The length of the insert may be less than the length between the bottom of the container and a surface of the lid facing the bottom of the container. In this regard, a space may be disposed between the top or bottom of the container and the insert. The space may be maintained by one or more anti-translation members, one or more anti-rotation members, or both. Where the walls of the container are tapered, the space may be maintained by engagement of the insert (e.g., by one or more anti-rotation members) to the container, where deeper location of the insert relative to the container may be precluded by a decreasing dimension in the cross-section of the tapered-wall container. Where the walls of the container comprise any portion with a smaller dimensioned cross-section relative to any other portions of the container, the space may be maintained by engagement of the insert (e.g., by one or more anti-rotation members) to the portion of the container having a smaller cross-section.

The device and/or kit may comprise a container. The container may function to receive the insert, capture extraneous plant matter, provide gripping surfaces for a user, or any combination thereof.

The container may have an open end and a closed end. A lid may be applied to the open end to seal the container.

The insert may be placed in the container and plant matter may be placed in the insert. A lid may seal the container. Upon rotation and/or gyration of the container, extraneous plant matter may collect within the open volume defined between the container and the insert.

The container may be a commercially available bucket, such as a 3.5-gallon or 5-gallon bucket. The container may or may not have a handle. The container may be a bag. The container and/or lid may be provided with the insert as a kit. The insert may be commercially provided to users and a suitable container may be purchased by the user.

The present disclosure contemplates that the insert may be used without the container. In this regard, extraneous plant matter may fall freely from the insert. Where the insert is used without the container, a lid or other suitable article may seal the open end of the insert.

The device and/or kit may comprise a lid. The lid may function to seal the container, cooperate with anti-rotation members, cooperate with anti-translation members, or any combination thereof, as described hereinbefore.

The lid may define a groove into which a rim of the container may fit. The groove and the rim of the container may friction-fit together. The container may comprise one or more projections extending around the outer surface of the container. The lid may comprise one or more projections on a portion of the lid that engages the outer surface of the container. In this regard, the portion of the lid with the one or more projections may elastically deform as the lid is pressed onto the container and the one or more projections of the lid engage a first side of the one or more projections of the container. The one or more projections of the lid may slide past the one or more projections of the container and elastically reform to engage a second side of the one or more projections of the container. The one or more projections of the lid and/or container may comprise a ramp and/or curvature to facilitate the one or more projections of the rim sliding along the one or more projections of the container.

The lid may engage the open end of the insert one or more anti-translation members, one or more anti-rotation members, or any combination thereof. The lid may apply a pressure to the insert. Where the inner diameter of the container is tapered from an end to an opposing end, the pressure applied may cause the anti-rotation members to apply a pressure to the inner surface of the container.

The lid may be provided with a window. The window may function to allow a user to view the contents of the container without removing the lid. The window may be fabricated from a polymer. The polymer may be optically transparent.

The explanations and illustrations presented herein are intended to acquaint others skilled in the art with the teachings, its principles, and its practical application. Those skilled in the art may adapt and apply the teachings in its numerous forms, as may be best suited to the requirements of a particular use. Accordingly, the specific embodiments of the present teachings as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The omission in the following claims of any aspect of subject matter that is disclosed herein is not a disclaimer of such subject matter, nor should it be regarded that the inventors did not consider such subject matter to be part of the disclosed inventive subject matter.

The disclosures of all articles and references, including patent applications and publications, are incorporated by reference for all purposes. Other combinations are also possible as will be gleaned from the following claims, which are also hereby incorporated by reference into this written description.

Plural elements or steps can be provided by a single integrated element or step. Alternatively, a single element or step might be divided into separate plural elements or steps.

The disclosure of “a” or “one” to describe an element or step is not intended to foreclose additional elements or steps.

While the terms first, second, third, etc., may be used herein to describe various elements, components, regions, layers, and/or sections, these elements, components, regions, layers, and/or sections should not be limited by these terms. These terms may be used to distinguish one element, component, region, layer, and/or section from another region, layer, and/or section. Terms such as “first”, “second”, and other numerical terms when used herein do not imply a sequence or order unless clearly indicated by the context. Thus, a first element, component, region, layer, and/or section discussed herein could be termed a second element, component, region, layer, and/or section without departing from the present teachings.

Spatially relative terms, such as “inner,” “outer,” “beneath,” “below,” “lower,” “above,” “upper,” and the like, may be used herein for ease of description to describe one element's relationship to other element(s), such as illustrated in the figures. Spatially relative terms may be intended to encompass different orientations of the device in use or operation in addition to the orientation depicted in the figures. For example, if the device is turned over, elements described as “below,” or “beneath” other elements would then be oriented “above” the other elements. Thus, the example term “below” can encompass both an orientation of “above” and “below.” The device may be otherwise oriented (e.g., rotated 90 degrees or at other orientations) and the spatially relative descriptors used herein are interpreted accordingly.

The terms “generally” or “substantially” to describe angular measurements may mean about +/−10° or less, about +/−5° or less, or even about +/−1° or less. The terms “generally” or “substantially” to describe angular measurements may mean about +/−0.01° or greater, about +/−0.1° or greater, or even about +/−0.5° or greater.

The terms “generally” or “substantially” to describe linear measurements, percentages, or ratios may mean about +/−10% or less, about +/−5% or less, or even about +/−1% or less. The terms “generally” or “substantially” to describe linear measurements, percentages, or ratios may mean about +/−0.01% or greater, about +/−0.1% or greater, or even about +/−0.5% or greater.

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

Unless otherwise stated, any numerical values recited herein include all values from the lower value to the upper value in increments of one unit provided that there is a separation of at least 2 units between any lower value and any higher value. As an example, if it is stated that the amount of a component, a property, or a value of a process variable such as, for example, temperature, pressure, time, and the like is, for example, from 1 to 90, from 20 to 80, or from 30 to 70, it is intended that intermediate range values such as 15 to 85, 22 to 68, 43 to 51, 30 to 32, etc., are within the teachings of this specification. Likewise, individual intermediate values are also within the present teachings. For values which are less than one, one unit is considered to be 0.0001, 0.001, 0.01, or 0.1 as appropriate. These are only examples of what is specifically intended and all possible combinations of numerical values between the lowest value and the highest value enumerated are to be considered to be expressly stated in this application in a similar manner. Unless otherwise stated, all ranges include both endpoints and all numbers between the endpoints.

The term “consisting essentially of” to describe a combination shall include the elements, ingredients, components, or steps identified, and such other elements ingredients, components or steps that do not materially affect the basic and novel characteristics of the combination. The use of the terms “comprising” or “including” to describe combinations of elements, ingredients, components, or steps herein also contemplates embodiments that consist essentially of the elements, ingredients, components, or steps.

### REFERENCE NUMERALS

10 Device
12 Insert
14 Container
16 Lid
18A Open end
18B Closed end
20 Outer surface
22 Inner surface
24 Openings
26 Agitation members
28 Anti-rotation member
30 Anti-translation member
32 Notch
34 Handle
l Longitudinal axis
t Transverse axis

What is claimed is:

1. A device for processing plant matter, the device comprising:
an insert having a generally tubular shape, the insert comprising:
one or more agitation members extending from an inner surface of the insert, and for promoting detachment of extraneous plant matter from subject plant matter;
a plurality of openings extending at least partially along the circumferential and longitudinal extent of the insert, the plurality of openings allowing detached extraneous plant matter to pass therethrough; and
one or more anti-rotation members extending from an outer surface of the insert;
wherein the insert is adapted, upon rotation and/or gyration relative to the longitudinal axis thereof, to cause the plant matter to contact the one or more agitation members.

2. The device according to claim 1, wherein the one or more anti-rotation members comprise a rim projecting from the outer surface of the insert.

3. The device according to claim 1, wherein the one or more anti-rotation members comprise at least two projections extending from the outer surface of the insert.

4. The device according to claim 1, wherein the insert has an open end allowing for the plant matter to be located within the insert; and wherein the one or more anti-rotation members are located on or proximate to the open end.

5. The device according to claim 1, wherein the one or more agitation members are scalloped regions extending from the inner surface of the insert; and wherein the scalloped regions extend at least partially from an end of the insert to an opposing end of the insert.

6. The device according to claim 5, wherein the insert comprises four scalloped regions generally equally spaced around the inner surface of the insert, and extending from the end of the insert to the opposing end of the insert.

7. The device according to claim 1, wherein the one or more agitation members are integrally molded to the insert.

8. The device according to claim 1, wherein the agitation members are fingers extending from the inner surface of the insert; and wherein the fingers are located at least partially along the inner surface from an end of the insert to an opposing end of the insert.

9. The device according to claim 8, wherein the fingers are located at least partially around the circumference of the inner surface.

10. The device according to claim 8, wherein the fingers are arranged in one or more rows along the length of the insert.

11. The device according to claim 1, wherein the device comprises a container for receiving the insert, and optionally the device comprises a lid to seal the container.

12. The device according to claim 11, wherein the one or more anti-rotation members engage the container and/or the lid to inhibit rotation of the insert within the container, maintain a gap between the insert and the container, or both.

13. The device according to claim 12, wherein the one or more anti-rotation members is a rim; and wherein the rim circumferentially engages an inner surface of the container.

14. The device according to claim 12, wherein the one or more anti-rotation members are projections extending from the outer surface of the insert device;
wherein the device comprises at least three of the anti-rotation members; and
wherein the at least three anti-rotation members are generally equally spaced around the insert.

15. The device according to claim 11, wherein the insert comprises one or more anti-translation members spanning a first gap and/or a second gap respectively between either or both opposing ends of the insert and the container; wherein the anti-translation members extend from either or both of the opposing ends of the insert; and wherein the one or more anti-translation members inhibit longitudinal axial translation of the insert within the container.

16. A kit for processing plant matter, the kit comprising:
a container;
an insert having a generally tubular shape and locatable into the container, the insert comprising:
one or more agitation members extending from an inner surface of the insert, and for promoting detachment of the plant matter;
a plurality of openings extending at least partially along the circumferential and longitudinal extent of the insert, the plurality of openings allowing the detached plant matter to pass therethrough; and
one or more anti-rotation members extending from an outer surface of the insert and engaging the container to inhibit rotation of the insert within the container, and maintain a gap between the insert and the container; and
optionally a lid to seal the container;
wherein the insert is adapted, upon rotation and/or gyration relative to the longitudinal axis thereof, to cause the plant matter to contact the one or more agitation members.

17. The kit according to claim 16, wherein the one or more agitation members are scalloped regions extending from the inner surface of the insert; and wherein the scalloped regions extend at least partially from an end of the insert to an opposing end of the insert.

18. The kit according to claim 17, wherein the insert comprises four scalloped regions integrally molded to the insert, generally equally spaced around the inner surface of the insert, and extending from the end of the insert to the opposing end of the insert.

19. The kit according to claim 16, wherein the one or more anti-rotation members are projections extending from the outer surface of the insert device; wherein the device comprises at least three of the anti-rotation members; and wherein the at least three anti-rotation members are generally equally spaced around the insert.

20. The device according to claim 16, wherein the device comprises one or more anti-translation members spanning a first gap and/or a second gap respectively between either or both opposing ends of the insert and the container; wherein the anti-translation members extend from either or both of the opposing ends of the insert; and wherein the one or more anti-translation members inhibit longitudinal axial translation of the insert within the container.
